SEQUENCE LISTING (16 sequences)

SEQ ID NO: 1 - DNA, 900 bp, Canis familiaris
atgcggtcacacacccagcatcccgggccttctgactcgccgggcgggaagatgtcatcattgccaagaagagcaaaagtaaaggtccaggctgtggtatccaaagatgaattctcttccttgtctgagttatcatctgcctctgaagaagatgacaaggaagatagtgcctgggagccccaaaagaaagtccccagaagtcgtaagcagcctgttgccaaggaatccaaaccaaagagggtgccacgggtgaagaagaccaccctacagatcaatgatggctcagaaggcgtggctgttaaggaggagctgaataactctgtggctattgctgatgttgatttggaagacaaaaaaattaaagtggataccgtgaagactttgaagacagcaaaggcaaaacggaagaattcaggtcagtcacctgcaggtcagaggatcaaaaagctraaasttgatgaagaatccaacagggccagcactgcagagacaccatccacaagcaccttgtgggaaggtgtgtgcaagaaggaagagagcgaagatgacttcacatttggtcagtcccctttaaagaaaatgaagactgaaacatgtcctcaggggcagcctgtgaggtttccagcaaatgcaaacaacattaaagaggaggtggaaatgaactgggacatagtacaggttttatctgagagaactaatattgaactttgggtatgtgccaacattattcgtctctttaatgatgataacacaattcccttcattatacggtatcgaaaagagcttattaataaccttgatgctgattccttgagagaagttcgacaaaccctagaggagctccgggctgttgcaaagaaggttcatagtacaatccaaaaaattaagaaggaagggaag

SEQ ID NO: 2 - DNA, 21 bp, Artificial
  primer
gctattgctgatgttgatttg

SEQ ID NO: 3 - DNA, 21 bp, Artificial
  primer
tgcagtgctggccctgttgga

SEQ ID NO: 4 - DNA, 21 bp, Artificial
  primer
ctcacggctcccagctcacgg

SEQ ID NO: 5 - DNA, 21 bp, Artificial
  primer
tgtgtgtgtgctcgggtgtag

SEQ ID NO: 6 - DNA, 24 bp, Artificial
  primer
ccattacgtataatcgcttctttt

SEQ ID NO: 7 - DNA, 20 bp, Artificial
  primer
cgcacacaatggttatcctg

SEQ ID NO: 8 - DNA, 20 bp, Artificial
  primer
aattgtatggctgggaccaa

SEQ ID NO: 9 - DNA, 20 bp, Artificial
  primer
accaccagaggacacggata

SEQ ID NO: 10 - DNA, 20 bp, Artificial
  primer
catgctgaacatctggtggt

SEQ ID NO: 11 - DNA, 20 bp, Artificial
  primer
gctggtctggatgattgtca

SEQ ID NO: 12 - DNA, 21 bp, Artificial
  primer
taaagtggataccgtgaagac

SEQ ID NO: 13 - DNA, 17 bp, Artificial
  primer
gcatttgctggaaacct

SEQ ID NO: 14 - DNA, 21 bp, Artificial
  primer
actctgtggctattgctgatg

SEQ ID NO: 15 - DNA, 20 bp, Artificial
  primer
gggactgaccaaatgtgaag

SEQ ID NO: 16 - DNA, 900 bp, Canis familiaris
cttcccttccttcttaattttttggattgtactatgaaccttctttgcaacagcccggagctcctctagggtttgtcgaacttctctcaaggaatcagcatcaaggttattaataagctcttttcgataccgtataatgaagggaattgtgttatcatcattaaagagacgaataatgttggcacatacccaaagttcaatattagttctctcagataaaacctgtactatgtcccagttcatttccacctcctctttaatgttgtttgcatttgctggaaacctcacaggctgcccctgaggacatgtttcagtcttcattttctttaaaggggactgaccaaatgtgaagtcatcttcgctctcttccttcttgcacacaccttcccacaaggtgcttgtggatggtgtctctgcagtgctggccctgttggattcttcatcaastttyagctttttgatcctctgacctgcaggtgactgacctgaattcttccgttttgcctttgctgtcttcaaagtcttcacggtatccactttaatttttttgtcttccaaatcaacatcagcaatagccacagagttattcagctcctccttaacagccacgccttctgagccatcattgatctgtagggtggtcttcttcacccgtggcaccctctttggtttggattccttggcaacaggctgcttacgacttctggggactttcttttggggctcccaggcactatcttccttgtcatcttcttcagaggcagatgataactcagacaaggaagagaattcatctttggataccacagcctggacctttacttttgctcttcttggcaatgatgacatcttcccgcccggcgagtcagaaggcccgggatgctgggtgtgtgaccgcat

(19) United States
(12) Patent Application Publication
(10) Pub. No.: US 2014/0141432 A1
(43) Pub. Date: May 22, 2014

(54) METHOD AND KIT FOR DIAGNOSING GLAUCOMA IN DOGS

(71) Applicants: Masaki Imayasu, Kasugai-shi (JP); Kissaou Tchedre, Kasugai-shi (JP); Nobuyuki Kanemaki, Sagamihara-shi (JP); Masahiro Sakaguchi, Sagamihara-shi (JP); Shinpei Kawarai, Sagamihara-shi (JP)

(72) Inventors: Masaki Imayasu, Kasugai-shi (JP); Kissaou Tchedre, Kasugai-shi (JP); Nobuyuki Kanemaki, Sagamihara-shi (JP); Masahiro Sakaguchi, Sagamihara-shi (JP); Shinpei Kawarai, Sagamihara-shi (JP)

(73) Assignees: School Corporation, Azabu Veterinary Medicine Educational Institution, Sagamihara-shi, Kanagawa (JP); MENICON CO., LTD., Nagoya-shi, Aichi (JP)

(21) Appl. No.: 14/232,107

(22) PCT Filed: Jul. 5, 2012
(86) PCT No.: PCT/JP2012/067173

§ 371 Date: Jan. 10, 2014

(30) Foreign Application Priority Data
Jul. 11, 2011 (JP) 2011-152745

(51) Int. Cl. C12Q 1/68

(52) U.S. Cl. CPC C12Q 1/6883
USPC 435/6.11

(57) ABSTRACT

The present invention provides a canine glaucoma-susceptibility gene and a method of using the gene.

Single nucleotide polymorphisms were analyzed and compared in glaucomatous dogs and normal dogs. Out of those polymorphic sites at which differences were observed between glaucomatous and normal dogs, two polymorphic sites of the Single Nucleotide Polymorphism Database (dbSNP) ID numbers rs22018513 and rs22018514 were found effective for diagnosis of glaucoma.

### TECHNICAL FIELD

The present invention relates to a method and a kit for diagnosing canine glaucoma.

### BACKGROUND ART

Canine glaucoma is a disease in which the flow of aqueous humor between the cornea and the lens is hindered, resulting in increased intraocular pressure which compresses the retina and the optic nerve to cause visual disturbances such as visual field constriction.

When the intraocular pressure rises, tired eyes and other symptoms occur such as headache, dizziness, and nausea. However, dogs have no subjective symptoms and, even if they feel discomfort, they can not appeal to their owners. Even their visual field has become constricted, they run as usual and show no change in their behavior. For this reason, canine glaucoma often can not be detected early, and in many cases glaucomatous dogs are brought to animal hospitals when their symptoms have advanced.

Although there is no prophylactic method for glaucoma, the progress of this disease can be inhibited when it is found at an early stage. Methods for this purpose include administration of eye drops (miotics, p-adrenergic blocking agents, prostaglandin analogs, etc.) or internal medications (osmotic diuretics, carbonic anhydrase inhibitors, etc.), surgery for reducing the production of aqueous humor, and surgery for increasing the drainage of aqueous humor, for example.

To date, diagnosis of canine glaucoma has been performed by tonometry, gonioscopy and ophthalmoscopy.

However, these methods are not sufficient for early detection of glaucoma. A method capable of diagnosing glaucoma more early and more reliably is required.

On the other hand, with respect to human glaucoma, search for disease-susceptibility genes has been pursued. For example, Mizuki et al. have completed a genome-wide association study (GWAS) on a Japanese population (patient: 305 samples; normal: 355 samples) with GeneChip 500 k Array Set, identifying two genes (SRBD1 and ELOVL5) which show marked association with glaucoma (Non-Patent Document No. 1).

### PRIOR ART LITERATURE

### Non-Patent Documents

Non-Patent Document No. 1: Ophthalmology 2010 July; 117(7):1331-8.e5. Epub 2010 Apr. 3

### DISCLOSURE OF THE INVENTION

### Problem for Solution by the Invention

It is an object of the present invention to find out a disease-susceptibility gene effective for diagnosing canine glaucoma and to provide a method of using the gene.

### Means to Solve the Problem

As a result of SNP analyses of a glaucomatous dog population and a normal dog population, the present inventors have found two SNPs which are especially effective for diagnosing glaucoma.

The present invention has been achieved based on these findings.

A summary of the present invention is as described below.
(1) A method of testing canine glaucoma, comprising identifying at least one nucleotide at a polymorphic site selected from the group consisting of a nucleotide at the polymorphic site of the Single Nucleotide Polymorphism Database (dbSNP) ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms.
(2) A reagent for testing canine glaucoma, comprising at least one component selected from the group consisting of the following components (a) and (b):
   (a) primers capable of amplifying a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms, and
   (b) probes capable of hybridizing to a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBDI-containing region in canine chromosome 10) and a nucleotide at a site of polymorphism in linkage disequilibrium with these polymorphisms.
(3) The reagent of (2) above, wherein the probe is immobilized to a solid phase.
(4) A test kit for canine glaucoma, comprising the reagent of (2) or (3) above.

### Effect of the Invention

According to the present invention, it has become possible to diagnose canine glaucoma more early and more accurately. In dogs which have been already affected by glaucoma, definitive diagnosis has become possible, enabling positive treatment. In dogs which have not been affected by glaucoma, prediction of onset has become possible, leading to a recommendation of frequent testing and, consequently, early detection of glaucoma.

The present specification encompasses the contents disclosed in the specification and/or the drawings of Japanese Patent Application No. 2011-152745 based on which the present patent application claims priority.

### BEST MODES FOR CARRYING OUT THE INVENTION

Hereinbelow, the present invention will be described in detail.

The present invention provides a method of testing canine glaucoma, comprising identifying at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms. The nucleotide sequence of SEQ ID NO: 1 is a sequence complementary to the nucleotide sequence of SEQ ID NO: 16. The nucleotide at the polymorphic site of dbSNP ID number rs22018513 is the 451^{st} nucleotide in the nucleotide sequence of SEQ ID NO: 16 present in an SRBD1-containing region in canine chromosome 10, and the nucleotide at the polymorphic site of dbSNP ID number rs22018514 is the 447^{th} nucleotide in the nucleotide sequence of SEQ ID NO: 16 present in an SRBD1-containing region in canine chromosome 10.

The polymorphism in linkage disequilibrium with the polymorphism of dbSNP ID number rs22018513 or rs22018514 may be a polymorphism in the LD block of dbSNP ID number rs22018513 or rs22018514. The polymorphism in linkage disequilibrium with the polymorphism of dbSNP ID number rs22018513 or rs22018514 may be other polymorphism such that a p value (indicator of significant difference between glaucomatous individuals and normal individuals) located in an SRBD1-containing region in canine chromosome 10 is less than 0.05.

When D′ value between SNPs is large, the SNPs are believed to be in linkage disequilibrium (Barrett J C, Fry B, Mailer J, Daly M J. Haploview: analysis and visualization of LD and haplotype maps. Bioinformatics. 2005; 21(2):263-265.; Gabriel S B, Schaffner S F, Nguyen H, et al. The structure of haplotype blocks in the human genome. Science. 2002; 296 (5576):2225-2229). Therefore, the polymorphism in linkage disequilibrium with the polymorphism of dbSNP ID number rs22018513 or rs22018514 is, for example, a polymorphism having a greater D′ value with respect to these polymorphisms.

LD blocks may be determined with Haploview software (Barrett J C, Fry B, Mailer J, Daly M J. Haploview: analysis and visualization of LD and haplotype maps. Bioinformatics. 2005; 21(2):263-265) by the method of Gabriel et al. (Gabriel S B, Schaffner S F, Nguyen H, et al. The structure of haplotype blocks in the human genome. Science. 2002; 296(5576):2225-2229).

In the present specification, single nucleotide polymorphism (SNP) is expressed with an “rs” number which is a reference SNP ID number in dbSNP (the Single Nucleotide Polymorphism Database of NCBI). Nucleotide positions are based on the genome database build2.1 of NCBI.

In the present specification, the term “test of glaucoma” is a concept encompassing those tests for judging whether the subjects' susceptibility to glaucoma is high or low and, for subjects which have already been affected by glaucoma, those tests for making a definitive diagnosis

rs22018513 is a polymorphism of adenine (A)/guanine (G) at nucleotide position 51049604 of canine chromosome 10. When the nucleotide at this site is G, it is judged that the subject is highly susceptible to glaucoma or is suffering from glaucoma.

rs22018514 is a polymorphism of cytosine (C)/guanine (G) at nucleotide position 51049600 of canine chromosome 10. When the nucleotide at this site is G, it is judged that the subject is highly susceptible to glaucoma or is suffering from glaucoma. When C is substituted with G, the encoded amino acid leucine is substituted with valine.

The SNP to be identified in the present invention may be either one SNP or a combination of SNPs. Either the sense strand or anti-sense strand of the relevant gene may be analyzed.

Further, the homozygosity or heterozygosity of the relevant polymorphism may sometimes be used as an indicator of diagnosis.

rs22018513 and rs22018514 exist in an exon of a gene, and rs22018514 is a mutation involving amino acid substitution. Polymorphisms in linkage disequilibrium with these polymorphisms may exist in an exon of a gene, in regions which regulate the expression of a gene (promoter region, enhancer region, etc.), in an intron of a gene, or in those regions either upstream or downstream of these genes which are in linkage disequilibrium therewith. Types of the polymorphism include, but are not limited to, a single nucleotide polymorphism and a polymorphism in which one to several tens of nucleotides (occasionally, several thousand nucleotides) are substituted, deleted, inserted, transferred or inverted.

In the test method of the present invention, identification of a nucleotide at a polymorphic site (i.e., determination of nucleotide species) may be performed by known methods of single nucleotide polymorphism analysis. Specific examples of methods of single nucleotide polymorphism analysis include, but are not limited to, sequence analysis, PCR, PCR-SSCP, hybridization, RFLP method, Taqman-PCR, the invader method, cycleave-PCR, and HRM method.

For identification of a nucleotide at a polymorphic site, genomic DNA may be extracted from a biological sample taken from a subject. Specific examples of the biological sample include, but are not limited to, blood, skin, oral mucosa, a tissue or cell collected or excised by surgery, and body fluids collected for the purpose of testing or the like (e.g., saliva, lymph, respiratory mucosa, sperm, sweat and urine). Preferably, the biological sample is whole blood collected from the vein of any of the limbs or the neck. It is possible to extract genomic DNA from biological samples using a commercial DNA extraction kit. Subsequently, a DNA comprising a polymorphic site is isolated, if necessary. The isolation of this DNA may typically be performed by PCR with genomic DNA or RNA as a template, using primers capable of hybridizing to the DNA comprising a polymorphic site.

The subjects of glaucoma test are those dog breeds which are predisposed to glaucoma. Specific examples of such breeds include, but are not limited to, Shiba Inu, American Cocker Spaniel, Chihuahua, Dachshund, Labrador Retriever, Maltese, Miniature Pinscher, Pug, Shih Tzu and Corgi.

Further, the present invention provides a reagent for testing canine glaucoma, comprising at least one component selected from the group consisting of the following components (a) and (b):
(a) primers capable of amplifying a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms, and
(b) probes capable of hybridizing to a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms.

The polymorphic site of dbSNP ID number rs22018513, the polymorphic site of dbSNP ID number rs22018514 and the polymorphic site in linkage disequilibrium with these polymorphic sites are as described above.

Further, the present invention provides a test kit for canine glaucoma, comprising the above-described reagent.

The primer and the probe which are components of the reagent of the present invention may be an oligonucleotide of at least 15 nucleotides in length. When the oligonucleotide is used as a primer, its length is usually 15 bp to 100 bp, preferably 17 bp to 30 bp. The primer is not particularly limited as long as it is capable of amplifying at least a part of a DNA comprising the above-described polymorphic site. The length of DNA which primers can amplify is usually 15-1000 bp, preferably 20-500 bp, more preferably 20-200 bp. When the oligonucleotide is used as a probe, its length is usually 5 by to 200 bp, preferably 7 bp to 100 bp, more preferably 7 bp to 50 bp. The probe is not particularly limited as long as it is capable of hybridizing to a DNA comprising the above-described polymorphic site.

In the present invention, the primer capable of amplifying a region comprising a polymorphic site may be one that is capable of starting the synthesis of a complementary strand toward the polymorphic site with a DNA comprising the polymorphic site being used as a template.

As examples of the primer which is a component of the reagent of the present invention, the following primer pairs consisting of a forward primer and a reverse primer may be enumerated.

Forward Primer:

(SEQ ID NO: 2)

5′-GCTATTGCTGATGTTGATTTG-3′
Reverse Primer:

(SEQ ID NO: 3)

5′-TGCAGTGCTGGCCCTGTTGGA-3′

The nucleotide sequence of SEQ ID NO: 2 is identical to a partial sequence of SEQ ID NO: 1 spanning from nucleotide positions 325 to 345.

The nucleotide sequence of SEQ ID NO: 3 is a sequence complementary to a partial sequence of SEQ ID NO: 1 spanning from nucleotide positions 466 to 486.

Forward Primer:

(SEQ ID NO: 12)

5′-TAAAGTGGATACCGTGAAGAC-3′
Reverse Primer:

(SEQ ID NO: 13)

5′-GCATTTGCTGGAAACCT-3′

The nucleotide sequence of SEQ ID NO: 12 is identical to a partial sequence of SEQ ID NO: 1 spanning from nucleotide positions 360 to 380.

The nucleotide sequence of SEQ ID NO: 13 is a sequence complementary to a partial sequence of SEQ ID NO: 1 spanning from nucleotide positions 616 to 632.

Forward Primer:

(SEQ ID NO: 14)

5′-ACTCTGTGGCTATTGCTGATG-3′
Reverse Primer:

(SEQ ID NO: 15)

5′-GGGACTGACCAAATGTGAAG-3′

The nucleotide sequence of SEQ ID NO: 14 is identical to a partial sequence of SEQ ID NO: 1 spanning from nucleotide positions 317 to 337.

The nucleotide sequence of SEQ ID NO: 15 is a sequence complementary to a partial sequence of SEQ ID NO: 1 spanning from nucleotide positions 549 to 568.

The primer has a nucleotide sequence capable of hybridizing to a region containing a polymorphic site. This nucleotide sequence may be a sequence identical or complementary to a region which is shifted by several nucleotides from a region comprising a polymorphic site identical or complementary to the nucleotide sequence as shown in any one of SEQ ID NO: 2 to 7.

In addition to a nucleotide sequence identical or complementary to the nucleotide sequence of a region comprising a polymorphic site, the primer may have any nucleotide sequence added thereto. For example, in primers for use in polymorphism analysis with IIs type restriction enzyme, a recognition sequence for IIs type restriction enzyme is added to the primer. Further, the primer may be modified. For example, primers labeled with a fluorescent substance or a binding affinity substance such as biotin or digoxin may be used.

In the present invention, the probe capable of hybridizing to a region comprising a polymorphic site may be any probe that is capable of hybridizing to a polynucleotide having the nucleotide sequence of the region comprising a polymorphic site. Preferred is a probe which specifically hybridizes to a DNA having the nucleotide sequence of the region comprising a polymorphic site. As used herein, the term “specifically hybridizes” means that cross-hybridization with DNA other than the DNA having the nucleotide sequence of the region comprising a polymorphic site is not significantly produced under ordinary hybridization conditions, preferably under stringent hybridization conditions (see, for example, the conditions disclosed in Sambrook et al., Molecular Cloning, Cold Spring Harbor Laboratory Press, New York, USA, the 2^{nd} edition, 1989). More specifically, a probe comprising a polymorphic site in its nucleotide sequence is preferable. Alternatively, depending on the method of analysis of the nucleotide at the polymorphic site, a probe may be designed so that its end corresponds to nucleotides adjacent to the polymorphic site. Therefore, a probe which does not comprise the polymorphic site in its nucleotide sequence but comprises a nucleotide sequence complementary to the region adjacent to the polymorphic site therein may also be enumerated as a preferable probe for use in the present invention.

Like primers, probes are allowed to have an alteration of its nucleotide sequence, an addition of nucleotide sequence, or any other modification. For example, a nucleotide sequence that constitutes FLAP and which is foreign to the genome is added to probes for use in the invader method. Such probes are also included in the probe of the present invention as long as they hybridize to a region comprising a polymorphic site. The nucleotide sequence constituting the probe of the present invention may be designed based on the nucleotide sequences of peripheral DNA regions around the polymorphic site of the present invention in the genome, depending on the analysis method to be employed.

A person of ordinary skill in the art would be able to design primers and probes, as appropriate for the analysis method to be employed, based on nucleotide sequence information on peripheral DNA regions comprising the polymorphic site. Nucleotide sequences constituting such primers and probed may be completely complementary to the relevant nucleotide sequence in the genome. Alternatively, those nucleotide sequences may be appropriately modified.

The primer and the probe may be synthesized by any method on the basis of the nucleotide sequences that constitute such primer and probe. A technique is known in which on the basis of a given nucleotide sequence, an oligonucleotide having that nucleotide sequence is synthesized. Further, in the synthesis of an oligonucleotide, it is possible to introduce any modification to the oligonucleotide using a nucleotide derivative modified with a fluorescent dye, biotin, or the like. A method is also known in which a fluorescent dye or the like is bound to a synthesized oligonucleotide.

The probe may be immobilized to a solid phase (DNA array). In a DNA array, a sample DNA or RNA is hybridized to a great number of probes located on the same plane. By scanning the plane, hybridization to each probe is detected. Since the reactions with a great number of probes can be observed simultaneously, a DNA array is useful for simultaneously analyzing many polymorphic sites, for example. As a method for immobilizing (arraying) nucleotides, an oligonucleotide-based array developed by Affymetrix may be mentioned. In an oligonucleotide array, the oligonucleotide is usually synthesized in situ. Known methods of in situ oligonucleotide synthesis are based on photolithography (Affymetrix), inkjet printing (Agilent), or BeadArray technology (Illumina), for example.

The oligonucleotide is constituted by a nucleotide sequence complementary to a region comprising the polymorphic site to be detected. The length of nucleotide probes to be bound to the basal plate is usually 10-100 bp, preferably 10-50 bp, more preferably 15-25 bp, when oligonucleotides are to be immobilized.

Samples for SNP detection by the DNA array method may be prepared from biological samples collected from individual subjects by methods well-known to a person of ordinary skill in the art. Biological samples are not particularly limited. For example, DNA samples may be prepared from genomic DNAs extracted from blood, tissue or cell such as skin or oral mucosa, tear, saliva, urine, stool or hair collected from individual subjects. A specific region of the genomic DNA is amplified with primers for amplifying a region comprising the polymorphic site to be evaluated. In the process, it is possible to amplify a plurality of regions simultaneously by multiplex PCR. Multiplex PCR is a PCR method in which a plurality of primer sets are used in the same reaction mixture. Multiplex PCR is useful for analyzing a plurality of polymorphic sites.

Generally, in the DNA array method, DNA samples are amplified by PCR, and the amplified products are labeled. For labeling amplified products, tagged primers are used. For example, genomic DNA is first amplified by PCR with a primer set that is specific for a region comprising the polymorphic site of interest. Subsequently, labeling PCR is performed using biotin-labeled primers to thereby synthesize biotin-labeled DNA. The thus synthesized biotin-labeled DNA is allowed to hybridize to oligonucleotide probes on the chip. The reaction mixture and conditions for hybridization may be adjusted appropriately depending on the length of the nucleotide probes to be immobilized to the solid phase and conditions such as reaction temperature. One of ordinary skill in the art would be able to design appropriate hybridization conditions. For detecting hybridized DNA, avidin labeled with a fluorescent dye is added. The DNA array is analyzed with a scanner and checked for the presence or absence of hybridization using fluorescence as an indicator.

One example of procedures to perform the test method of the present invention using the DNA array method is as follows. Briefly, a polymorphic site-comprising DNA sample as prepared from a subject and a nucleotide probe-immobilized solid phase are provided. Then, the DNA sample is brought into contact with the solid phase. Subsequently, by detecting the DNA hybridizing to the immobilized nucleotide probe, the nucleotide species at the polymorphic site is determined.

In the present invention, the term “solid phase” means a material to which nucleotides can be immobilized. The solid phase is not particularly limited as long as it is capable of immobilizing nucleotides. Specific examples of the solid phase include, but are not limited to, microplate wells, plastic beads, magnetic particles, and basal plates. Generally, basal plates used in the DNA array technology may advantageously be used as a solid phase. In the present specification, the term “basal plate” means a tabular material to which nucleotides can be immobilized. Further, in the present invention, the term nucleotide encompasses oligonucleotides and polynucleotides.

In addition to the above-described methods, the allele-specific oligonucleotide (ASO) hybridization method may be used for detection of a nucleotide at a specific site. An ASO is constituted by a nucleotide sequence that hybridizes to a region comprising a polymorphic site to be detected. If, in the process of hybridizing an ASO to a sample DNA, mismatches occur at the polymorphic site because of the polymorphism, the efficiency of hybrid formation will decrease. Mismatches may typically be detected by Southern blotting or by a method that makes use of the tendency of a special fluorescent reagent to undergo fluorescence quenching on account of intercalation into the gap of the hybrid. It is also possible to detect mismatches by the ribonuclease A mismatch cleavage method.

The reagent and the kit of the present invention may comprise various enzymes, enzyme substrates, buffers, and the like depending on the method of identification of nucleotides. Examples of such enzymes include, but are not limited to, DNA polymerases, DNA ligases or IIs restriction enzymes which are necessary for the various analysis methods enumerated above as methods for identification of nucleotides. As buffers, those which are suitable for maintaining the activity of the enzymes used in these analyses are appropriately selected. Enzyme substrates that can be used may include, for example, a substrate for synthesizing a complementary strand.

Further, a control in which the nucleotide species at a polymorphic site of interest is well-defined may be added as an attachment to the reagent and the kit of the present invention. As a control, a genomic DNA which has been preliminarily identified for the nucleotide species at a polymorphic site of interest or a fragment of such genomic DNA may be used. A genomic DNA extracted from cells may be attached as a control. Alternatively, a cell or cell fraction may be attached to the reagent or the kit as a control, from which the user may extract a genomic DNA. When a cell is used as a control, the result with the control may provide a proof that extraction of genomic DNA has been performed accurately. Alternatively, a DNA consisting of a nucleotide sequence comprising a polymorphic site of interest may be used as a control. Specifically, YAC vector or BAC vector comprising a DNA derived from a genome in which the nucleotide species at a polymorphic site of interest has been identified may be used as a control. Alternatively, a DNA fragment of several tens of by to several hundred by in length (corresponding to a polymorphic site of interest) may be selectively cut out from a genomic DNA and inserted into a vector, which may also be used as a control.

### EXAMPLES

Hereinbelow, the present invention will be described in more detail with reference to the following Example. However, the present invention is not limited to this Example.

### Example 1

### 1. Diagnosis of Canine Glaucoma and Purification of DNA

1) Shiba Inu dogs (pure line) which visited the Azabu University Veterinary Teaching Hospital were locally anesthetized by administering Benoxil to both of their eyes.
2) The intraocular pressure (IOP) of both eyes was measured with a TONO-PEN. Dogs with IOP of 24 mmHg or more were judged glaucomatous, and dogs with IOP of less than 24 mmHg were judged normal. (glaucoma: 47 dogs; normal: 34 dogs)
3) Blood (1 mL) was collected from the jugular vein, followed by purification of DNA by the DNA whole blood kit/spin method (Fuji Film).
4) The concentration and purity of the resultant DNA were determined with GeneQuant Pro (GE Health Science).

### 2. Determination of DNA Nucleotide Sequence

1) PCR was performed using TaKaRa Ex Taq (Takara Bio Inc.). PCR conditions were as described below.

<Reagents>

| | | |
|---|---|---|
| Taq polymerase | 0.25 | μL |
| 10xBuffer | 5 | μL |
| dNTPs | 4 | μL |
| Template | 2 | μL |
| Forward Primer | 1 | μL |
| (5′-GCTATTGCTGATGTTGATTTG-3′) | | |
| (SEQ ID NO: 2) | | |
| Reverse Primer | 1 | μL |
| (5′-TGCAGTGCTGGCCCTGTTGGA-3′) | | |
| (SEQ ID NO: 3) | | |
| H₂O | 11.75 | μL |
| Total Volume | 25 | μL |

<Amplification Conditions>

(94° C. for 5 min) 1 cycle

(98° C. for 10 sec, 55° C. for 30 sec, 72° C. for 1 min) 40 cycles

(72° C. for 7 min) 1 cycle

2) The PCR product was electrophoresed on 11 agarose gel and stained with ethidium bromide to confirm detection of the PCR product as a single band. The band was recovered in a 1.5 mL tube.
3) TE buffer (50 μL) was added to the tube, which was then frozen at −80° C. for more than 3 hours.
4) The sample was thawed, and the sample DNA was purified with Qiagen DyeEx 2.0 Spin Kit (QIAGEN).
5) Cycle sequencing was performed under the following conditions.

### <Reagents>

| | | |
|---|---|---|
| 5xBuffer | 4 | μL |
| Big Dye | 6 | μL |
| Template | 6 | μL |
| Forward Primer | 1 | μL (or Reverse Primer 1 μL) |
| H₂O | 3 | μL |
| Total Volume | 20 | μL |

<Sequencing Conditions>

(95° C. for 2 min) 1 cycle

(94° C. for 1 min, 50° C. for 30 sec, 72° C. for 4 min) 30 cycles

(72° C. for 7 min)

6) The sample DNA was purified again with Qiagen EyeEx 2.0 Spin Kit and dried.
7) High Dye Mix (Life Technologies) (20 μL) was added to the sample DNA. The resultant mixture was treated at 90° C. for 3 min and then ice-cooled for 5 min.
8) The sample was transferred into a sequencing tube, followed by analysis of the nucleotide sequence with ABI 310 Sequencer (Life Technologies).
9) According to the nucleotide sequences of the two SNPs (rs22018513 and rs22018514) in canine SRBD1 gene, the genotypes of the samples (non-risk homo, hetero, and risk homo) were judged as shown in the Table below.

TABLE 1

| SNP | Primer | Non-Risk Homo | Hetero | Risk Homo |
|---|---|---|---|---|
| rs22018513 | Forward | AA | AG | GG |
| | Reverse | TT | CT | CC |
| rs22018514 | Forward | CC | CG | GG |
| | Reverse | GG | CG | CC |

The results of analysis of genetic polymorphisms on 47 glaucomatous Shiba Inu dogs and 34 normal Shiba Inu dogs are shown in Table 2.

With respect to the polymorphic site of rs22018513, the risk allele frequency in the glaucoma group was 78.7%, while that frequency in the normal group was 55.9%. From comparison of these two groups, it was found that the risk to develop glaucoma is 2.92 times as high when a dog has the risk allele; a significant difference (p<0.01) was observed by chi-square test.

With respect to the polymorphic site of rs22018514, the risk allele frequency in the glaucoma group was 39.4%, while that frequency in the normal group was 17.6%. From comparison of these two groups, it was found that the risk to develop glaucoma is 3.03 times as high when a dog has the risk allele; a significant difference (p<0.01) was observed by chi-square test.

From these results, it was estimated that the risk to develop glaucoma will be nearly 9 times as high when a dog has the risk allele at both polymorphic sites of rs22018513 and rs22018514.

The results of analysis of genetic polymorphisms on 22 glaucomatous Shih Tzus and 28 normal Shih Tzus are also shown in Table 2.

With respect to the polymorphic site of rs22018513, the risk allele frequency in the glaucoma group was 97.7%, while that frequency in the normal group was 85.7%. From comparison of these two groups, it was found that the risk to develop glaucoma is 7.17 times as high when a dog has the risk allele; a significant difference (p<0.05) was observed by chi-square test.

TABLE 2
Shiba Inu SRBD1

| | | | | Risk | |
|---|---|---|---|---|---|
| | Non-Risk | Hetero | Risk | Allele (G) | Statistical |
| rs22018513 | AA | AG | GG | Frequency | Processing |
| Glaucoma | 1 | 18 | 28 | 78.7% | Chi-square |
| Normal | 5 | 20 | 9 | 55.9% | test: P < 0.01 |
| | | | | | Odds ratio: 2.92 |
| | | | | Risk | |
| | Non-Risk | Hetero | Risk | Allele (G) | Statistical |
| rs22018514 | CC | CG | GG | Frequency | Processing |
| Glaucoma | 18 | 21 | 8 | 39.4% | Chi-square |
| Normal | 22 | 12 | 0 | 17.6% | test: P < 0.01 |
| | | | | | Odds ratio: 3.03 |

Shih Tzu SRBD1

| | | | | Risk | |
|---|---|---|---|---|---|
| | Non-Risk | Hetero | Risk | Allele (G) | Statistical |
| rs22018513 | AA | AG | GG | Frequency | Processing |
| Glaucoma | 0 | 1 | 21 | 97.7% | Chi-square |
| Normal | 0 | 8 | 20 | 85.7% | test: P < 0.05 |
| | | | | | Odds ratio: 7.17 |
| | | | | Risk | |
| | Non-Risk | Hetero | Risk | Allele (G) | Statistical |
| rs22018514 | CC | CG | GG | Frequency | Processing |
| Glaucoma | 0 | 5 | 17 | 88.6% | Chi-square |
| Normal | 0 | 6 | 22 | 89.3% | test: N.S. |
| | | | | | Odds ratio: 0.93 |

The same detection procedures were applied to other gene mutations, except for using the following pairs of forward primer and reverse primer.

For rs24048794 detection
Forward Primer:

(SEQ ID NO: 4)

5′-CTCACGGCTCCCAGCTCACGG-3′
Reverse Primer:

(SEQ ID NO: 5)

5′-TGTGTGTGTGCTCGGGTGTAG-3′
For rs24048796 detection
Forward Primer:

(SEQ ID NO: 4)

5′-CTCACGGCTCCCAGCTCACGG-3′
Reverse Primer:

(SEQ ID NO: 5)

5′-TGTGTGTGTGCTCGGGTGTAG-3′
For rs24048798 detection
Forward Primer:

(SEQ ID NO: 4)

5′-CTCACGGCTCCCAGCTCACGG-3′
Reverse Primer:

(SEQ ID NO: 5)

5′-TGTGTGTGTGCTCGGGTGTAG-3′
For rs8571991 detection
Forward Primer:

(SEQ ID NO: 6)

5′-CCATTACGTATAATCGCTTCTTTT-3′
Reverse Primer:

(SEQ ID NO: 7)

5′-CGCACACAATGGTTATCCTG-3′
For rs8643563 detection
Forward Primer:

(SEQ ID NO: 8)

5′-AATTGTATGGCTGGGACCAA-3′
Reverse Primer:

(SEQ ID NO: 9)

5′-ACCACCAGAGGACACGGATA-3′
For rs22202438 detection
Forward Primer:

(SEQ ID NO: 10)

5′-CATGCTGAACATCTGGTGGT-3′
Reverse Primer:

(SEQ ID NO: 11)

5′-GCTGGTCTGGATGATTGTCA-3′

The results of analysis of genetic polymorphisms of 10 glaucomatous Shiba Inu dogs and 10 normal Shiba Inu dogs are shown in Table 3.

With respect to OPTC1 gene, the minor allele frequency at the polymorphic site of rs24048794 was 10% in both glaucoma group and normal group. No statistically significant difference was observed. At the polymorphic site of rs24048796, the minor allele frequency was 0% in both glaucoma group and normal group. No statistically significant difference was observed. At the polymorphic site of rs24048798, the minor allele frequency was 0% in both glaucoma group and normal group. No statistically significant difference was observed.

With respect to CYP1B1 gene, the minor allele frequency at the polymorphic site of rs8571991 was 0% in both glaucoma group and normal group. No statistically significant difference was observed.

With respect to ELOVL5 gene, the minor allele frequency at the polymorphic site of rs8643563 was 0% in both glaucoma group and normal group. No statistically significant difference was observed. At the polymorphic site of rs22202438, the minor allele frequency was 45% in both glaucoma group and normal group. No statistically significant difference was observed.

From the results described so far, it was shown that the polymorphic sites of rs24048794, rs24048796, rs24048798, rs8571991, rs8643563 and rs22202438 do not correlate with the risk to develop glaucoma.

TABLE 3
OPTC1

| | Major | | Minor | | |
|---|---|---|---|---|---|
| | Allele | | Allele | Minor | |
| | Homo | Hetero | Homo | Allele (C) | Statistical |
| rs24048794 | AA | AC | CC | Frequency | Processing |
| Glaucoma | 9 | 0 | 1 | 10% | Chi-square |
| Normal | 9 | 0 | 1 | 10% | test: No |
| | | | | | significant |
| | | | | | difference |
| | Major | | Minor | | |
| | Allele | | Allele | Minor | |
| | Homo | Hetero | Homo | Allele (T) | Statistical |
| rs24048796 | CC | CT | TT | Frequency | Processing |
| Glaucoma | 10 | 0 | 0 | 0% | Chi-square |
| Normal | 10 | 0 | 0 | 0% | test: No |
| | | | | | significant |
| | | | | | difference |
| | Major | | Minor | | |
| | Allele | | Allele | Minor | |
| | Homo | Hetero | Homo | Allele (G) | Statistical |
| rs24048798 | AA | AG | GG | Frequency | Processing |
| Glaucoma | 10 | 0 | 0 | 0% | Chi-square |
| Normal | 10 | 0 | 0 | 0% | test: No |
| | | | | | significant |
| | | | | | difference |

CYP1B1

| | Major | | Minor | | |
|---|---|---|---|---|---|
| | Allele | | Allele | Minor | |
| | Homo | Hetero | Homo | Allele (G) | Statistical |
| rs8571991 | (—)(—) | (—)G | GG | Frequency | Processing |
| Glaucoma | 10 | 0 | 0 | 0% | Chi-square |
| Normal | 10 | 0 | 0 | 0% | test: No |
| | | | | | significant |
| | | | | | difference |

ELOVL5

| | Major | | Minor | | |
|---|---|---|---|---|---|
| | Allele | | Allele | Minor | |
| | Homo | Hetero | Homo | Allele (T) | Statistical |
| rs8643563 | (—)(—) | (—)T | TT | Frequency | Processing |
| Glaucoma | 10 | 0 | 0 | 0% | Chi-square |
| Normal | 10 | 0 | 0 | 0% | test: No |
| | | | | | significant |
| | | | | | difference |

| | Major | | Minor | | |
|---|---|---|---|---|---|
| | Allele | | Allele | Minor | |
| | Homo | Hetero | Homo | Allele (G) | Statistical |
| rs22202438 | AA | AG | GG | Frequency | Processing |
| Glaucoma | 2 | 7 | 1 | 45% | Chi-square |
| Normal | 3 | 5 | 2 | 45% | test: No |
| | | | | | significant |
| | | | | | difference |

(—) represents deletion.

All publications, patents and patent applications cited herein are incorporated herein by reference in their entirety.

### INDUSTRIAL APPLICABILITY

The present invention is applicable to veterinary medicine and diagnosis.

### SEQUENCE LISTING FREE TEXT

### <SEQ ID NO: 1>

SEQ ID NO: 1 shows a nucleotide sequence of 900 nucleotides in length comprising the polymorphic site of rs22018513 at position 450 (r=A/G) and the polymorphic site of rs22018514 at position 454 (r=C/G).

### <SEQ ID NO: 2>

SEQ ID NO: 2 shows the nucleotide sequence of a forward primer used for detecting the polymorphic sites of rs22018513 and rs22018514.

### <SEQ ID NO: 3>

SEQ ID NO: 3 shows the nucleotide sequence of a reverse primer used for detecting the polymorphic sites of rs22018513 and rs22018514.

### <SEQ ID NO: 4>

SEQ ID NO: 4 shows a nucleotide sequence of a forward primer used for detecting the polymorphic sites of rs24048794, rs24048796, and rs24048798.

### <SEQ ID NO: 5>

SEQ ID NO: 5 shows a nucleotide sequence of a reverse primer used for detecting the polymorphic sites of rs24048794, rs24048796, and rs24048798.

### <SEQ ID NO: 6>

SEQ ID NO: 6 shows the nucleotide sequence of a forward primer used for detecting the polymorphic site of rs8571991.

### <SEQ ID NO: 7>

SEQ ID NO: 7 shows the nucleotide sequence of a reverse primer used for detecting the polymorphic site of rs8571991.

### <SEQ ID NO: 8>

SEQ ID NO: 8 shows the nucleotide sequence of a forward primer used for detecting the polymorphic site of rs8643563.

### <SEQ ID NO: 9>

SEQ ID NO: 9 shows the nucleotide sequence of a reverse primer used for detecting the polymorphic site of rs8643563.

### <SEQ ID NO: 10>

SEQ ID NO: 10 shows the nucleotide sequence of a forward primer used for detecting the polymorphic site of rs22202438.

### <SEQ ID NO: 11>

SEQ ID NO: 11 shows the nucleotide sequence of a reverse primer used for detecting the polymorphic site of rs22202438.

### <SEQ ID NO: 12>

SEQ ID NO: 12 shows the nucleotide sequence of a forward primer which may be used for detecting the polymorphic sites of rs22018513 and rs22018514.

### <SEQ ID NO: 13>

SEQ ID NO: 13 shows the nucleotide sequence of a reverse primer which may be used for detecting the polymorphic sites of rs22018513 and rs22018514.

### <SEQ ID NO: 14>

SEQ ID NO: 14 shows the nucleotide sequence of a forward primer which may be used for detecting the polymorphic sites of rs22018513 and rs22018514.

### <SEQ ID NO: 15>

SEQ ID NO: 15 shows the nucleotide sequence of a reverse primer which may be used for detecting the polymorphic sites of rs22018513 and rs22018514.

### <SEQ ID NO: 16>

SEQ ID NO: 16 shows a sequence complementary to the nucleotide sequence of SEQ ID NO: 1. This is a nucleotide sequence of 900 nucleotides in length comprising the polymorphic site of rs22018513 at position 451 (y=T/C) and the polymorphic site of rs22018514 at position 447 (s=G/C).

1. A method of testing canine glaucoma, comprising identifying at least one nucleotide at a polymorphic site selected from the group consisting of a nucleotide at the polymorphic site of the Single Nucleotide Polymorphism Database (dbSNP) ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms.

2. A reagent for testing canine glaucoma, comprising at least one component selected from the group consisting of the following components (a) and (b):
(a) primers capable of amplifying a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms, and
(b) probes capable of hybridizing to a region containing at least one nucleotide at a polymorphic site selected from the group consisting of a nucleotide at the polymorphic site of dbSNP ID number rs22018513 (the 450^{th} nucleotide in the nucleotide sequence of SEQ ID NO: 1 present in an SRBD1-containing region in canine chromosome 10), a nucleotide at the polymorphic site of dbSNP ID number rs22018514 (the 454^{th} nucleotide in the nucleotide sequence of SEQ ID NO: 1 present in an SRBD1-containing region in canine chromosome 10) and a nucleotide at a site of polymorphism in linkage disequilibrium with these polymorphisms.

3. The reagent according to claim 2, wherein the probe is immobilized to a solid phase.

4. A test kit for canine glaucoma, comprising the reagent according to claim 2 or 3.
